(19) United States
(12) Patent Application Publication
(10) Pub. No.: US 2024/0041281 A1
(43) Pub. Date: Feb. 8, 2024

(54) Autonomous Cleaning Robot System and Method

(71) Applicant: Zhenwu Huang, Las Vegas, NV (US)

(72) Inventor: Zhenwu Huang, Las Vegas, NV (US)

(21) Appl. No.: 17/817,639

(22) Filed: Aug. 4, 2022

(51) Int. Cl. A47L 7/00; A47L 9/00; A47L 9/04; A47L 9/14; A47L 9/28; A47L 11/24; A47L 11/30; A47L 11/40; A61L 2/10; A61L 2/24; A61L 2/26

(52) U.S. Cl. CPC A47L 7/0061; A47L 9/009; A47L 9/0477; A47L 9/0472; A47L 9/0488; A47L 7/0033; A47L 9/1409; A47L 9/2805; A47L 9/2852; A47L 9/2847; A47L 9/2842; A47L 11/24; A47L 11/305; A47L 11/4011; A47L 11/4022; A47L 11/4027; A47L 11/4041; A47L 11/4038; A47L 11/405; A47L 11/4083; A47L 11/4088; A61L 2/10; A61L 2/24; A61L 2/26; A47L 2201/04; A47L 2201/06; A61L 2202/17; A61L 2202/14; A61L 2202/11

(57) ABSTRACT

An autonomous cleaning robot includes a main body including a main mounting frame and an outer shell, a drive wheel assembly, a collision mitigation mechanism, a sweeping and vacuum assembly, a cleaning fluid applicator and cleaning roller assembly, a germicidal ultraviolet light disinfection mechanism, the germicidal ultraviolet light disinfection mechanism capable of disinfecting both cleaning fluid and a cleaning surface, and a main controller unit. The robot uses germicidal ultraviolet radiation to both disinfect a cleaning surface as well as to disinfect used and/or recycled cleaning fluid. A method for cleaning a floor involves activating the robot; checking fluid levels; acquiring imagery of the space to be cleaned, dispensing cleaning fluid, recycling recovered and excess cleaning fluid, irradiating used cleaning fluid, irradiating a cleaning surface, moving across a cleaning area, recording a travel path, traveling across areas not previously traveled across, and powering down when finished.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

The invention relates generally to the field of cleaning robots. More specifically, the present invention relates to an autonomous cleaning robot system and method which recycles cleaning fluid and employs germicidal ultraviolet radiation to disinfect both cleaning fluid as well as the surface to be cleaned.

Autonomous robot floor cleaning devices are known in the art. Such devices are configured to clean hard floor surfaces such as tile, wood, and concrete floors, and to freely move from one surface type to the other unattended and without interrupting the cleaning process. Known devices typically include an autonomous locomotion mechanism, a brush mechanism and a vacuum mechanism.

Germicidal techniques including the use of various types of sanitizers have been developed to combat the contamination of surfaces, and are well known in the art. Numerous methods inventions have been created and implemented to clean and sanitize surfaces, the surrounding air, and liquids. Among the most popular methods of sanitizing both surfaces and the surrounding air is the use of chemical sanitizers.

Chemical sanitizers for surfaces involve the introduction of a chemical mixture onto the surface. Chemical sanitizers ranging from alcohols to chlorine-based sanitizers have been used for well over 100 years. In recent years, the use of alcohol-based solutions has become prevalent among surface cleaners and disinfectants. Though this method of disinfecting air has proven effective, many chemical sanitizers can irritate respiratory and integumentary systems. Finally, chemical based sanitizer systems, when used in a reservoir, lose their effectiveness after use.

As an alternative to chemical sanitizers, the use of ultraviolet irradiation devices has become widely used. Ultraviolet sanitizing devices essentially consist of a device where ultraviolet lights irradiate surfaces and the surrounding space. Such ultraviolet irradiation has proven itself highly effective in killing pathogens, and several portable devices have been created.

Newer autonomous cleaning robots provide surface and floor clearing in the form of collecting loose particulates, as well as applying a cleaning fluid to a floor. However, there exists no compact autonomous cleaning robot with a system for recycling cleaning fluid in such a manner which enables a cleaning robot to clean a larger surface area. Moreover, there is currently no teaching of an autonomous floor cleaning robot capable of employing germicidal ultraviolet radiation to disinfect both the surface to be cleaned and the cleaning fluid. Presently, there exists a need for such an autonomous cleaning robot system with a system for recycling cleaning fluid in such a manner which enables a cleaning robot to clean a larger surface area. Additionally, there exists a need for an autonomous cleaning robot capable of using germicidal ultraviolet radiation to both disinfect used cleaning fluid as well as disinfect a cleaning surface.

### SUMMARY

The present invention is directed an improved autonomous cleaning robot system and method which employs a fluid recycling system and germicidal ultraviolet radiation technology to filter, disinfect and re-use used cleaning fluid. Furthermore, the improved autonomous cleaning robot system uses germicidal ultraviolet radiation technology to disinfect surfaces. Such an improved autonomous cleaning robot is designed to clean and disinfect floors without having to frequently change out cleaning fluid. Moreover, the improved autonomous cleaning robot system produces improved results in the form of a cleaner and sanitized surface.

The invention, at its essence, includes a main body including a main mounting frame and an outer shell; a drive wheel assembly; a collision mitigation mechanism; a sweeping and vacuum assembly; a cleaning fluid applicator and cleaning roller assembly; a germicidal ultraviolet light disinfection mechanism, said germicidal ultraviolet light disinfection mechanism capable of disinfecting both cleaning fluid and a cleaning surface; and a main controller unit. The use of germicidal ultraviolet radiation to both disinfect a cleaning surface as well as to disinfect used and/or recycled cleaning fluid.

More specifically, the invention includes a main body including a main mounting frame and an outer shell; a drive wheel assembly; a collision mitigation mechanism; a sweeping and vacuum assembly; a cleaning fluid applicator and roller assembly; a germicidal ultraviolet light disinfection mechanism capable of disinfecting both cleaning fluid and a cleaning surface; a cleaning fluid recycling assembly; and a main controller unit. The autonomous cleaning robot adds a cleaning fluid recycling system which works in tandem with the sweeping mechanism, the vacuuming mechanism, and the mopping mechanism. A wringing roller wrings cleaning fluid from a roller brush. The used cleaning fluid is directed into a receiving tray capable of accommodating dirty cleaning fluid. The used cleaning solution is filtered by a filter mechanism, is disinfected by an ultraviolet light assembly, and the disinfected and filtered cleaning fluid is pumped back to a fluid reservoir to continue circulating in the mopping mechanism.

Embodiments of the invention include sweeping and vacuum assembly which comprises a sweeper having two counterrotating brushes, a dust collector, a vacuum cannister, and a suction tube connecting the dust collector with the vacuum cannister. The twin counterrotating brushes sweep debris into a dust collector where the vacuum draws any dust and debris into a cannister.

The cleaning fluid applicator and roller assembly includes a cleaning fluid reservoir; a cleaning fluid outlet pipeline; and a roller brush assembly for cleaning and mopping a floor. The cleaning fluid recycling assembly includes a wringing roller mechanism for wringing a roller brush assembly for cleaning and mopping a floor, the roller mechanism for wringing the roller brush assembly for cleaning and mopping a floor being engageable with the roller brush assembly; a fluid receiving tray; a filtering mechanism for cleaning fluid; and a cleaning fluid pump. Such a mechanism allows for the efficient dispensation, application, recycling and filtering of cleaning fluid.

The germicidal ultraviolet light disinfection mechanism of the cleaning roller assembly includes at least one UV-C disinfecting lamp. In embodiments of the invention, the UV-C disinfecting lamp is configured to apply germicidal ultraviolet radiation to the contents inside the fluid reservoir as well as configured to apply germicidal ultraviolet radiation to the surface to be cleaned and disinfected.

Use of the autonomous is designed to be simple. As a method for cleaning a floor using an autonomous cleaning robot system involves activating the autonomous cleaning robot; checking of cleaning fluid levels; the robot system acquiring imagery of the space to be cleaned; the robot activating the sweeping and vacuum assembly; the robot dispensing cleaning fluid through a cleaning fluid applicator and roller assembly; the robot recycling recovered and excess cleaning fluid; the robot activating the germicidal ultraviolet light disinfection mechanism to irradiate recovered and excess cleaning fluid, and irradiating a cleaning surface; the autonomous cleaning robot moving across the area or space to be cleaned; the autonomous cleaning robot recording its travel path; the autonomous cleaning robot traveling across areas not previously traveled across; and housing and powering down the autonomous cleaning robot when the autonomous cleaning robot completes the task of cleaning the surface to be cleaned.

### BRIEF DESCRIPTION OF THE DRAWINGS

The invention directed by way of example, and not by way of limitation, in the figures of the accompanying drawings and in which like reference numerals refer to similar elements and in which:

FIG. 1 is a perspective view of an autonomous cleaning robot in accordance with an embodiment of the invention;

FIG. 2 is a perspective view of an autonomous cleaning robot system without an outer cover in accordance with an embodiment of the invention;

FIG. 3 is a perspective view of the underside of an autonomous cleaning robot in accordance with an embodiment of the invention;

FIG. 4 is an exploded perspective view of an autonomous cleaning robot system in accordance with an embodiment of the invention;

FIG. 5 is a perspective view of the mopping mechanism of an autonomous cleaning robot system in accordance with an embodiment of the invention;

FIG. 6 is a perspective view of the spray plate of an autonomous cleaning robot system in accordance with an embodiment of the invention;

FIG. 7 is an exploded perspective view of the mopping mechanism of an autonomous cleaning robot system in accordance with an embodiment of the invention;

FIG. 8 is a perspective sectional view of the mopping mechanism of an autonomous cleaning robot system in accordance with an embodiment of the invention;

FIG. 9 is a perspective view of a roller brush mounting frame assembly of an autonomous cleaning robot system in accordance with an embodiment of the invention;

FIG. 10 is a perspective view of the vacuum and sweeping assembly of an autonomous cleaning robot system in accordance with an embodiment of the invention;

FIG. 11 is a perspective sectional view of the vacuum and sweeping assembly of an autonomous cleaning robot system in accordance with an embodiment of the invention;

FIG. 12 is an exploded perspective view of the vacuum and sweeping assembly of an autonomous cleaning robot system in accordance with an embodiment of the invention;

FIG. 13 is a perspective view of the collision mitigation mechanism of an autonomous cleaning robot system in accordance with an embodiment of the invention;

FIG. 14 is a block diagram of a typical computer system that, when appropriately configured or designed, may serve as a computer system for which the main controller unit of the autonomous cleaning robot, and the components thereof, may be embodied;

FIG. 15 is a block diagram depicting the general architecture and functionality of an autonomous cleaning robot in accordance with an embodiment of the invention; and

FIG. 16 is a flow diagram of a method for operating an exemplary autonomous cleaning robot system in accordance with an embodiment of the invention; and

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

References to “one embodiment,” “one variant,” “an embodiment,” “a variant,” “various embodiments,” “numerous variants,” etc., may indicate that the embodiment(s) of the invention so described may include particular features, structures, or characteristics. However, not every embodiment or variant necessarily includes the particular features, structures, or characteristics. Further, repeated use of the phrase “in one embodiment,” or “in an exemplary embodiment,” or “a variant,” or “another variant,” do not necessarily refer to the same embodiment although they may. A description of an embodiment with several components in communication with each other does not imply that all such components are required. On the contrary, a variety of optional components are described to illustrate the wide variety of possible embodiments and/or variants of the present invention.

A “computer” may refer to one or more apparatus and/or one or more systems that are capable of accepting a structured input, processing the structured input according to prescribed rules, and producing results of the processing as output. Examples of a computer may include: a personal computer (PC); a stationary and/or portable computer; a computer having a single processor, a computer having multiple processors, or a computer having multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer; a personal digital assistant (PDA); a portable telephone; a portable smartphone; wearable devices such as smartwatches; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

The term “processor” may refer to any device or portion of a device that processes electronic data from registers and/or memory to transform that electronic data into other electronic data that may be stored in registers and/or memory. A “computing platform” may comprise one or more processors.

A “computer monitor” or “display” is an output device that displays information in pictorial form. A monitor usually comprises the visual display, circuitry, casing, and power supply. The display device in modern monitors is typically a thin film transistor liquid crystal display (TFT-LCD) with LED backlighting having replaced cold-cathode fluorescent lamp (CCFL) backlighting. Monitors are typically connected to computers via VGA, Digital Visual Interface (DVI), S-Video, HDMI, DisplayPort, Thunderbolt, low-voltage differential signaling (LVDS) or other proprietary and/or integrated connectors and signals.

It will be readily understood by persons skilled in the art that the various methods and algorithms described herein may be implemented by appropriately programmed computers and computing devices. Typically, a processor (e.g., a microprocessor) will receive instructions from a memory or memory-like device, and execute those instructions, thereby performing a process defined by those instructions. Further, programs that implement such methods and algorithms may be stored and transmitted using a variety of known media.

“Software” may refer to prescribed rules and/or instructions used to operate a computer. Examples of software may include code segments in one or more computer-readable languages; graphical and or/textual instructions; applets; pre-compiled code; interpreted code; compiled code; and computer programs. An operating system or “OS” is software that manages computer hardware and software resources and provides common services for computer programs.

Certain embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including object-oriented programming languages and/or conventional procedural programming languages, and/or programming languages or other compilers, assemblers, interpreters or other computer languages or platforms.

A “computer system” may refer to a system having one or more computers, where each computer may include a computer-readable medium employing software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

Terms such as, but not limited to, “Sanitizer,” or “Disinfectant” refers to a substance or device for killing or reducing levels of pathogenic microorganisms.

“Ultraviolet germicidal irradiation” or “UVGI” is a germicidal technique where ultraviolet radiation is used to kill or inactivate microorganisms. Ultraviolet radiation is mutagenic and harmful to bacteria, viruses and other microorganisms, with short-wavelength ultraviolet radiation considered to be “germicidal” at wavelengths between 100-280 nanometers.

As is well known to those skilled in the art, many careful considerations and compromises typically must be made when designing the optimal manufacture or commercial implementation of such an autonomous cleaning robot system and method. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary autonomous cleaning robot system and method will now be described in detail with reference to embodiments thereof as illustrated in the accompanying drawings. Unless otherwise indicated illustrations in the figures are not necessarily drawn to scale.

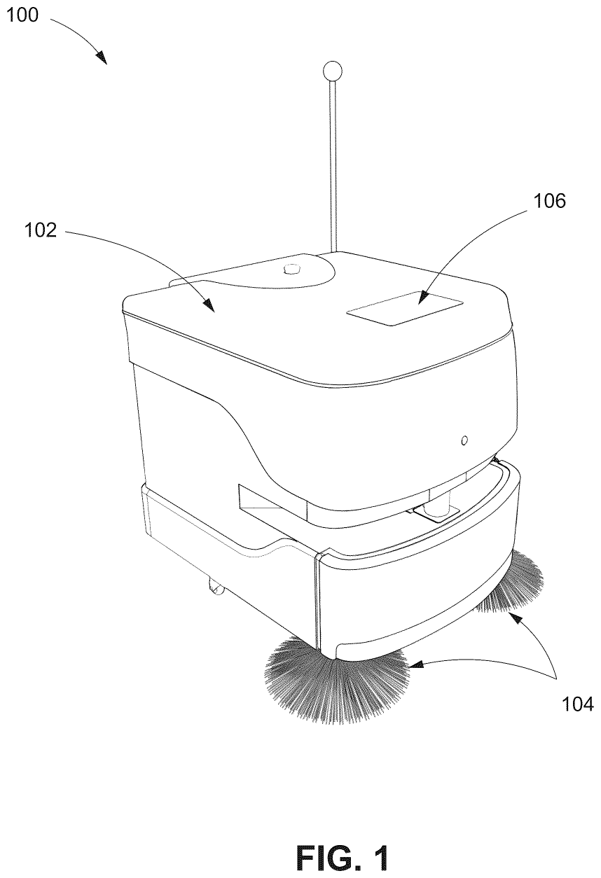

FIG. 1 is a perspective view of an autonomous cleaning robot system in accordance with an embodiment of the invention. In this view, the autonomous cleaning robot system 100 includes a main body which includes an outer shell 102. In embodiments of the invention, the outer shell 102 can be removable from a frame unit. can attach to a chassis so as to create a single unit.

Visible from the outside of the system is a display interface 106. Such a display interface can be coupled with a main controller unit including at least one processor and memory having computer readable instructions capable of controlling the individual components of the autonomous cleaning robot system. The autonomous cleaning robot employs front sensors which include visual cameras and radar sensors known in the art. The radar sensors are the main or primary sensors and can perform functions such as scanning and obstacle avoidance. However, the radar is installed at a certain height, and the area below the radar installation height cannot be scanned. In one embodiment of the invention, each camera intervenes to scan for unknown risks such as obstacles and stairs on the ground. Data gathered from both radar and the cameras is stored in memory, where calculations are made and instructions delivered to the drive wheels to achieve optimal obstacle avoidance.

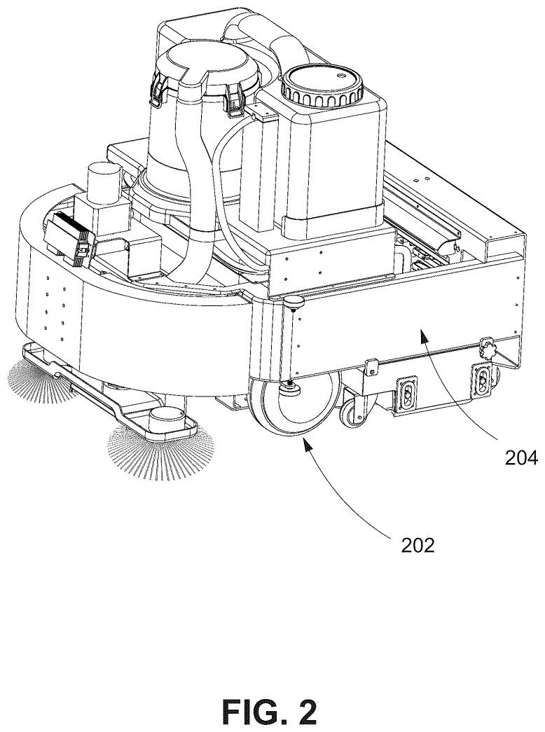

FIG. 2 is a perspective view of an autonomous cleaning robot system without an outer cover in accordance with an embodiment of the invention. In this view, a drive wheel assembly consisting of two drive wheels 202 govern the motion of the autonomous cleaning robot. A main mounting frame 204 is used to attach the main components to the frame and robot while providing a means for absorbing impacts. Persons having skill in the art will appreciate that a main mounting frame can consist of one or more cross members as well as one or more frame rails. A main mounting frame is generally used to provide structural stability while protecting the components from impact forces. A collision mitigation mechanism consisting of impact absorbing bumpers and a reinforced frame allows for greater durability while not sacrificing mobility and utility.

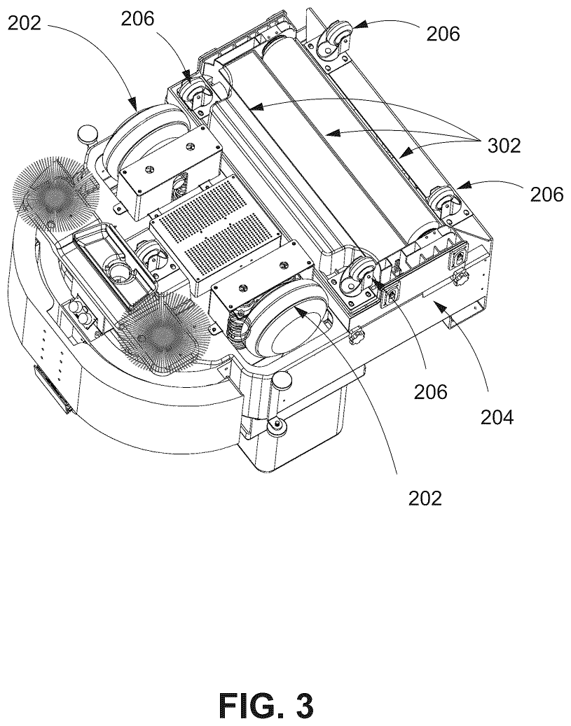

FIG. 3 is a perspective view of the underside of an autonomous cleaning robot in accordance with an embodiment of the invention. In this view, a drive wheel assembly consisting of two drive wheels 202 govern the motion of the autonomous cleaning robot. A main mounting frame 204 is used to attach the main components to the frame while providing a means for absorbing impacts. Four caster assemblies 206 provide support for the autonomous cleaning robot while facilitating motion and turning. Each caster assembly is configured to rotate freely about a substantially vertical axis as the autonomous cleaning robot moves over a cleaning surface by the drive wheels 202. Accordingly, each caster assembly is self-aligning with respect to the direction of robot transport. Persons having skill in the art will appreciate that a main mounting frame can consist of one or more cross members as well as one or more frame rails. The main mounting frame is used to provide structural stability while protecting internal and external components from impact forces. A collision mitigation mechanism consisting of impact absorbing bumpers and a reinforced frame allows for greater durability while not sacrificing mobility and utility.

One or more apertures 302 in the underside of the autonomous cleaning robot allow for germicidal ultraviolet radiation to travel from a light source located internally to the cleaning surface. Such a configuration allows for a germicidal light assembly to irradiate and disinfect cleaning fluid, cleaning brushes and other components while simultaneously being able to irradiate and disinfect a cleaning surface such as a floor. Persons having skill in the art will readily appreciate that various UV-C lighting devices can be used and positioned to achieve such results.

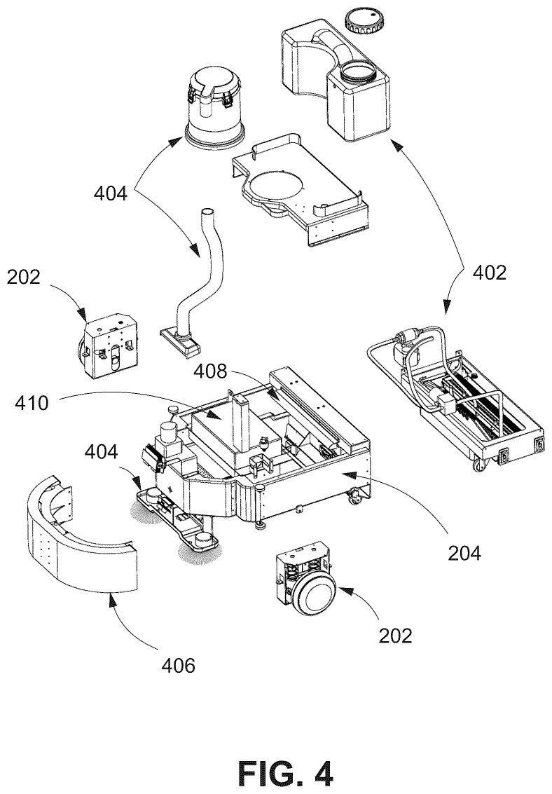

FIG. 4 is an exploded perspective view of an autonomous cleaning robot system in accordance with an embodiment of the invention. In this view, the invention comprises a drive wheel assembly 202, a main mounting frame 204, a cleaning roller assembly including a germicidal light mechanism 402, a sweeping and vacuum assembly 404, and a front bumper unit 406. Persons skilled in the art will appreciate that the components connect to the main mounting frame 204 to form a functioning unit.

In embodiments of the invention, an ultraviolet light disinfection mechanism 408 is installed on a rear structure of the main mounting frame 204. When the robot begins a cleaning cycle, the main controller unit will control the ultraviolet light disinfection mechanism to turn on and sterilize and/or disinfect the circulating cleaning fluid and cleaning roller assembly. The ultraviolet light disinfection mechanism is capable of projecting germicidal light through one or more openings or apertures in the chassis or underside or undercarriage where it can also irradiate the ground and disinfect the cleaning surface. Such a unique combination allows for both sanitized cleaning fluid and a disinfected cleaning surface. In one embodiment of the invention, the ultraviolet light disinfection system employs a tube lamp with a length of 287 mm, power is 8 W, and operates at a wavelength of 254 nm. Such a wavelength belongs to short-wave UV-C, which is easily absorbed by the DNA of the organism and is the strongest sterilizer. However, other embodiments of the invention can employ LED UV germicidal lamps. By way of example, and not limitation, use of one or more LED lamps in various configurations can provide a more optimal disinfection system while requiring less space than conventional tube lamps. Moreover, LED lamps may draw less electricity from any batter used.

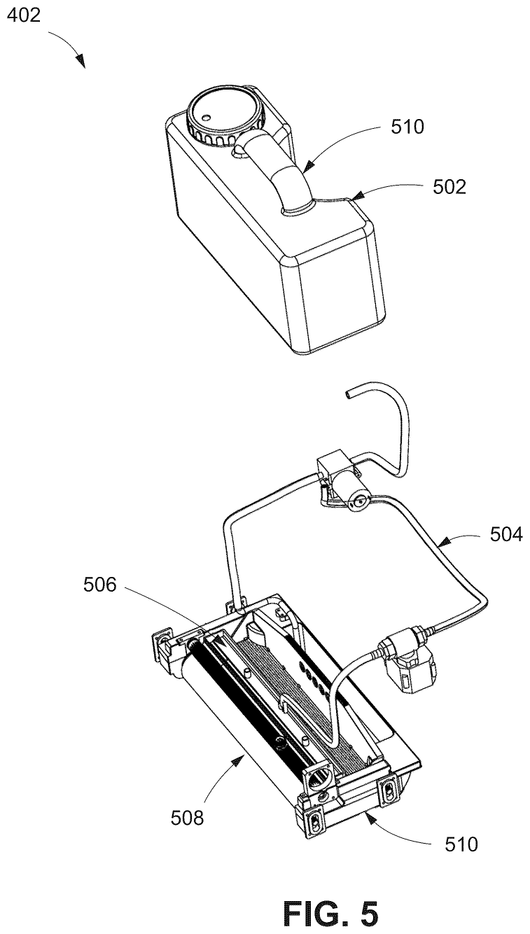

FIG. 5 is a perspective view of the cleaning roller assembly 402 of an autonomous cleaning robot system in accordance with an embodiment of the invention. In this view, the cleaning roller assembly mechanism comprises a cleaning fluid reservoir 502, a cleaning fluid outlet pipeline 504, a spray plate assembly 506, a roller brush 508 and a rolling brush mounting frame 510 for roller mopping. In this embodiment, the roller brush 508 is cylindrical as a whole, and is made of a synthetic material such as, but not limited to, a polyurethane plastic. The roller brush is designed to be replaced at regular intervals. Persons having skill in the art will understand that there are numerous means to attach the roller brush 508 to the cleaning roller assembly mechanism. By way of example, and not limitation, screws and buckles at the roller mounting points can be used to securely attach the cleaning roller to the cleaning roller assembly mechanism. A user need only remove screws to remove the old roller brush 508 and install a new one.

The cleaning fluid reservoir 502 supplies cleaning fluid to the roller brush 508 through a cleaning fluid outlet pipeline 504. The roller brush 508 is rotatably mounted on the rolling brush mounting frame 510. The spray plate assembly 506 is located above the roller brush 508. Positioned above the spray plate 506 is a clean fluid distribution well 512, the bottom of the clean fluid distribution well 512 is provided with two equalizing dams 514. The cleaning fluid reservoir 502 has a handle 518, so that a user can lift the cleaning fluid reservoir 502 to add or remove or change a suitable cleaning fluid or clean the fluid reservoir 502.

The spray plate 506 has a flow channel inside where the clean fluid distribution well 512 is filled with cleaning fluid. The flow channel connects with the bottom of the spray plate 506 and includes a plurality of spray ports. The flow channel connects with the spray ports, and the spray ports are in communication with the fluid outlet pipeline 504.

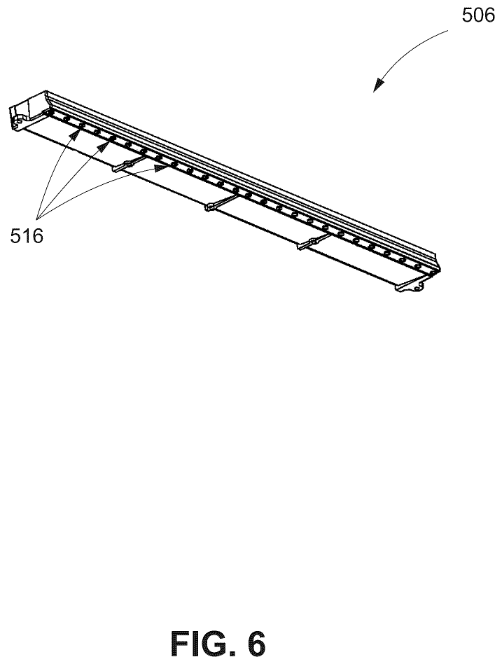

FIG. 6 is a perspective view of the spray plate of an autonomous cleaning robot system in accordance with an embodiment of the invention. A plurality of spray ports 516 are arranged along the length of the spray plate 506, so that a suitable cleaning fluid can be sprayed onto the roller brush 508 evenly. In one embodiment of the invention, there are a total of 26 spray ports. The spray plate is made of a plastic material. There is a layer of filter sponges inside. After filtering the dirty water, the clean water is sprayed out through the nozzle. The filter sponges can be made from any suitable material capable of filtering cleaning fluid.

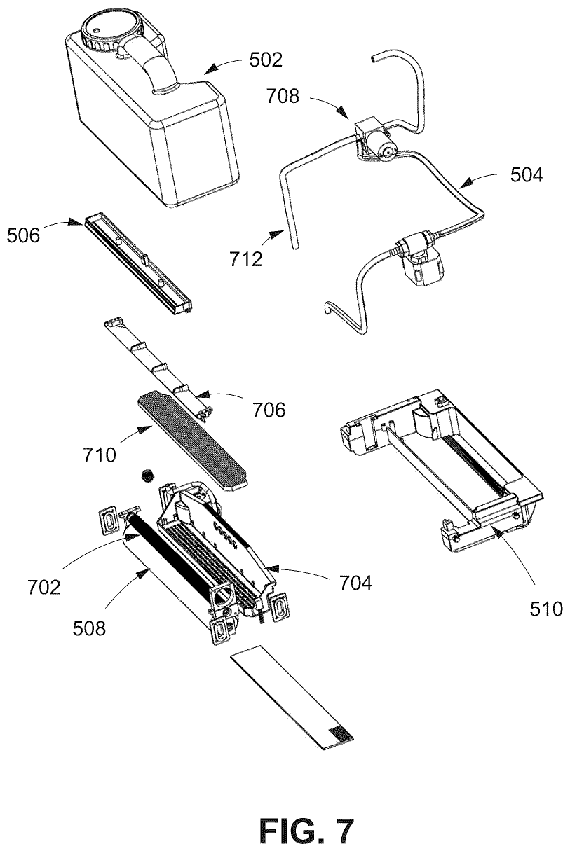

FIG. 7 is an exploded perspective view of the cleaning roller assembly of an autonomous cleaning robot system in accordance with an embodiment of the invention. In this view, the cleaning roller assembly mechanism comprises a cleaning fluid reservoir 502, a cleaning fluid outlet pipeline 504, a spray plate assembly 506, a roller brush 508 and a rolling brush mounting frame 510 for rolling mopping. In this embodiment, the roller brush 508 is cylindrical as a whole, and is made of a synthetic material such as, but not limited to, a polyurethane plastic. The cleaning fluid reservoir 502 supplies water to the roller brush 508 through the cleaning fluid outlet pipeline 504. The roller brush 508 is rotatably mounted on the rolling brush mounting frame 510. The spray plate assembly 506 is located above the roller brush 508.

The cleaning roller assembly includes a fluid circulation system which consists of a wringing roller 702 for wringing or applying pressure to the roller brush 508. The fluid circulation system includes a fluid receiving tray 704 for accommodating the dirty fluid squeezed or wrung out from the roller brush 508. The system further includes a deflector 706. Filter mechanism for filtering sewage, UVC disinfection lamp and fluid pump 708.

The wringing roller 702 and the deflector 706 are fixedly installed on the rolling brush mounting frame 510. The wringing roller 702 is positioned close to the outer periphery of the roller brush 508, with the axes of the two rollers being parallel. The fluid receiving tray 704 is located below the roller brush 508 with the wringing roller 702 positioned above the roller brush 508. In an embodiment of the invention, the wringing roller 702 is cylindrically shaped, with its surface including protrusions assuming different shapes, through which a frictional force is applied to the roller brush 508.

The deflector 706 is inclined (with a certain angle to the horizontal plane) and is arranged between the fluid receiving tray 704 and the roller brush 508, and its lower side is connected to the fluid receiving tray 704, so that contaminated or used cleaning fluid can be guided from the roller brush 508 to the fluid receiving tray 704.

In order to prevent water from flowing into the bearing and affecting the bearing, the two ends of the above-mentioned roller brush 508 and the wringing roller 702 are embedded within a fluid retaining wall. The roller brush mounting frame 510 is a skeleton-type metal frame, which is more convenient to assemble and can better ensure the positional accuracy of the rotating shaft. A filtering mechanism is installed between the fluid receiving tray 704 and the fluid pump 708. Dirty cleaning fluid or solution recovered in the fluid receiving tray 704 is filtered by a filtering mechanism and is then returned to the fluid tank 502 through the water pump 708.

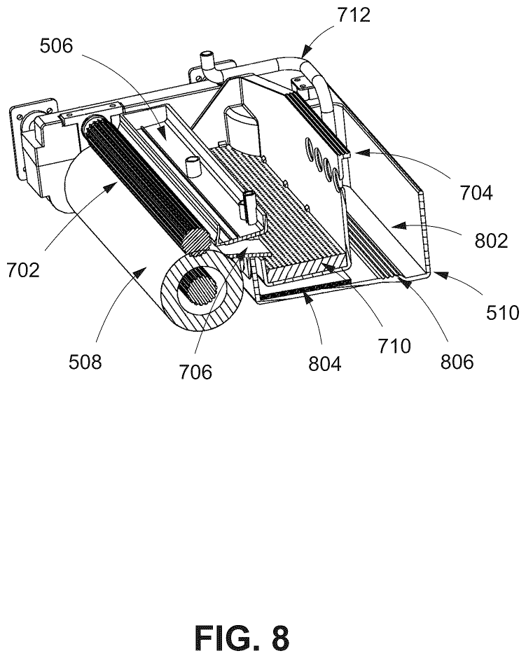

FIG. 8 is a perspective sectional view of the cleaning roller assembly of an autonomous cleaning robot system in accordance with an embodiment of the invention. In this view, the cleaning roller assembly mechanism comprises a spray plate assembly 506, a roller brush 508 and a rolling brush mounting frame 510 for rolling mopping. In this embodiment, the roller brush 508 is cylindrical, and is made of a synthetic material such as, but not limited to, a polyurethane plastic. The fluid reservoir 502 supplies water to the roller brush 508 through the water outlet pipeline fluid outlet pipeline 504. The roller brush 508 is rotatably mounted on the rolling brush mounting frame 510. The spray plate assembly 506 is located above the roller brush 508.

The cleaning roller assembly includes a fluid circulation system which consists of a wringing roller 702 for wringing or applying pressure to the roller brush 508. The fluid circulation system includes a fluid receiving tray 704 for accommodating the dirty fluid squeezed or wrung out from the roller brush 508. The system further includes a deflector 706. The deflector 706 is inclined (with a certain angle to the horizontal plane) and is positioned between the fluid receiving tray 704 and the roller brush 508, and its lower side is connected to the fluid receiving tray 704, so that used cleaning fluid can be guided from the roller brush 508 to the fluid receiving tray 704. In other words, the deflector is positioned between the fluid receiving tray and the roller brush in such a manner that used cleaning fluid can be guided from the roller brush into the fluid receiving tray.

In order to prevent cleaning fluid from flowing into the roller brush bearings and fouling the bearings, the two ends of the roller brush 508 and the two ends of wringing roller 702 are protected by a fluid retaining wall. The roller brush mounting frame 510 is a skeleton-type metal frame, which is more convenient to assemble and can better ensure the positional accuracy of the rotating shaft. A filtering mechanism is installed between the fluid receiving tray 704 and the fluid pump 708. Dirty cleaning fluid or solution recovered in the fluid receiving tray 704 is filtered by a filtering mechanism and is then returned to the fluid tank 502 through the cleaning fluid pump 708.

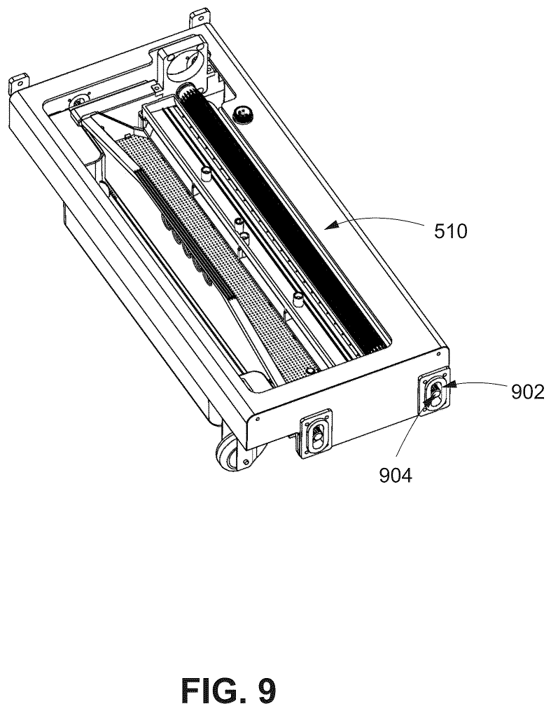

FIG. 9 is a perspective view of a roller brush mounting frame assembly of an autonomous cleaning robot system in accordance with an embodiment of the invention. In this view, the roller brush mounting frame 510 includes a suspension frame having at least two openings 902 located on the side walls of the roller brush mounting frame 510. The mounting shafts 904 extend out respectively, and the mounting shafts 904 penetrate into the opening 902. When the mopping module encounters an uneven ground during mopping, the mounting shaft 904 moves up and down in the opening 902 to ensure that the roller brush is in full contact with the ground, so as to avoid missing brushes and mopping. In various embodiments of the invention, elastic components can also be embedded in the opening 902 to increase the adhesion of the roller brush to the ground and ensure an optimum cleaning effect. In some embodiments of the invention, the roller brush assemblies have spring structures on both sides, which can allow the mopping module to apply force to the roller brush assembly for cleaning and mopping a floor to adapt to varying surface conditions, so as to clean up. In addition, the roller itself is also a polymer elastic material.

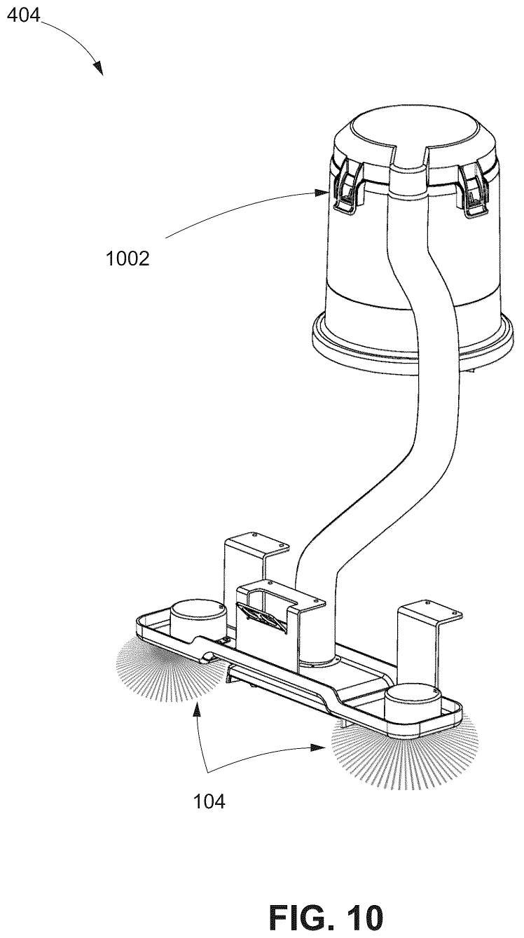

FIG. 10 is a perspective view of the vacuum and sweeping assembly of an autonomous cleaning robot system in accordance with an embodiment of the invention. In this view, the vacuuming and sweeping mechanism includes two cleaning brushes 104 and a vacuum cleaner 1002. The two cleaning brushes 104 are symmetrically installed on both sides of the bottom of the cleaning robot in a rotatable manner. In this embodiment, each cleaning brush 104 is claw-shaped, and is made from an elastic material such as, but not limited to, a plastic or polymer. However, any suitable material for brushing mechanisms will be understood by persons skilled in the art. When cleaning, the two cleaning brushes 104 both rotate inwardly (that is, the cleaning brush 104 on the left rotates clockwise, and the cleaning brush 104 on the right rotates counterclockwise), and presses against the ground. Each cleaning brush 104 deforms into a plane structure parallel to, and in contact with, the ground. Each claw-shaped cleaner 104 is capable of providing a temporary accommodation space for debris and trash to prevent said debris and trash from being thrown out or scattered. In one embodiment of the invention, each brush motor is a 12 volt, 10 watt, 0.6 A motor capable of producing a rotary speed of 10600 RPM. However, the brush speed will typically range from 10-600 RPM depending on need. Persons having skill in the art will appreciate that memory storing computer readable instructions when executed by a controller unit or processor or microprocessor can govern speeds of the motors.

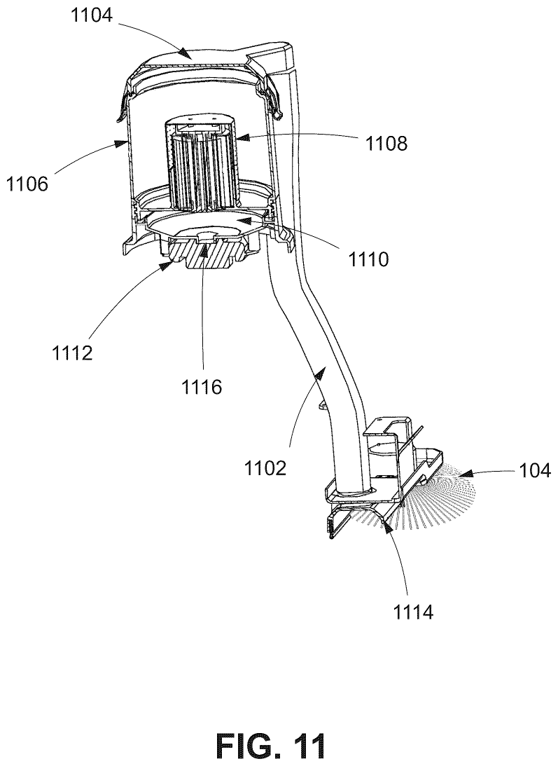

FIG. 11 is a perspective sectional view of the vacuum and sweeping assembly of an autonomous cleaning robot system in accordance with an embodiment of the invention. In this view, the vacuuming and sweeping mechanism includes a vacuum cleaner body and a vacuum pipe 1102. The vacuum pipe 1102 includes a conventional section and a vacuum port section, and the vacuum port section is close to the cleaning brush 104. A suction hood 1114 for guiding dust and garbage into the dust suction pipe 1102 is provided at the mouth.

The main body of the vacuum cleaner includes an upper cover 1104, a barrel 1106, a dust filter component 1108, a chassis 1110 and a vacuum cleaner motor assembly 1112. The upper cover 1104 covers the barrel body 1106 in a detachable manner, and the barrel body 1106 covers the chassis 1110 in a detachable manner. In an embodiment of the invention, a professional strength vacuum motor and vacuum assembly 1112, powered by a battery, and controlled by a main controller is used.

In an embodiment of the invention, the upper cover 1104 is connected to the top end of the barrel body 1106 by a locking means known and appreciated in the art, and the bottom end of the barrel body 1106 is connected to the chassis 1110 by means of a screw connection. The barrel body 1106 has a hollow cavity for accommodating trash and debris and an installation cavity for installing the dust filter part 1108. The wall of the installation cavity is provided with a number of air inlet holes, and the air in the accommodating cavity enters the dust filter part 1108 through the air inlet holes. Inside, the interior of the dust filter part 1108 has an air outlet, the chassis 1110 is provided with an air outlet 1116 at the position corresponding to the air outlet channel, the fan 1112 is located under the chassis 1110, and the air inlet of the fan 1112 faces the air outlet 1116. The top end is mounted on the upper cover 1104.

In this embodiment, the horizontal section of the accommodating cavity is annular, the installation cavity is located at the center of the accommodating cavity, and the dust filter member 1108 is in the shape of a hollow cylinder as a whole. Air, dust and trash are sucked into the accommodating cavity of the barrel 1106 from the dust suction port, and the air flows into the dust filter part 1108 under the suction force of the fan 1112 for filtering, and then is discharged through the air outlet channel and the exhaust port 1116.

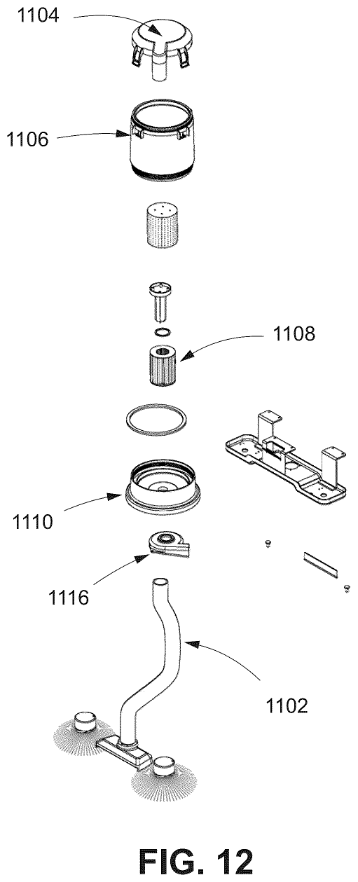

FIG. 12 is an exploded perspective view of the vacuum and sweeping assembly of an autonomous cleaning robot system in accordance with an embodiment of the invention. In this view, the vacuum and sweeping assembly includes an upper cover 1104, a barrel 1106, a dust filter component 1108, a chassis 1110 and a fan 1112. The upper cover 1104 covers the barrel body 1106 in a detachable manner, and the barrel body 1106 covers the chassis 1110 in a detachable manner.

In an embodiment of the invention, the upper cover 1104 is connected to the top end of the barrel body 1106 by a locking means known and appreciated in the art, and the bottom end of the barrel body 1106 is connected to the chassis 1110 by means of a screw connection. The barrel body 1106 has a hollow cavity for accommodating trash and debris and an installation cavity for installing the dust filter part 1108. The wall of the installation cavity is provided with a number of air inlet holes, and the air in the accommodating cavity enters the dust filter part 1108 through the air inlet holes. Inside, the interior of the dust filter part 1108 has an air outlet, the chassis 1110 is provided with an air outlet 1116 at the position corresponding to the air outlet channel, the fan 1112 is located under the chassis 1110, and the air inlet of the fan 1112 faces the air outlet 1116. The top end is mounted on the upper cover 1104.

In this embodiment, the horizontal section of the accommodating cavity is annular, the installation cavity is located at the center of the accommodating cavity, and the dust filter member 1108 is in the shape of a hollow cylinder as a whole. Air, dust and trash are sucked into the accommodating cavity of the barrel 1106 from the dust suction port, and the air flows into the dust filter part 1108 under the suction force of the fan 1112 for filtering, and then is discharged through the air outlet channel and the exhaust port 1116.

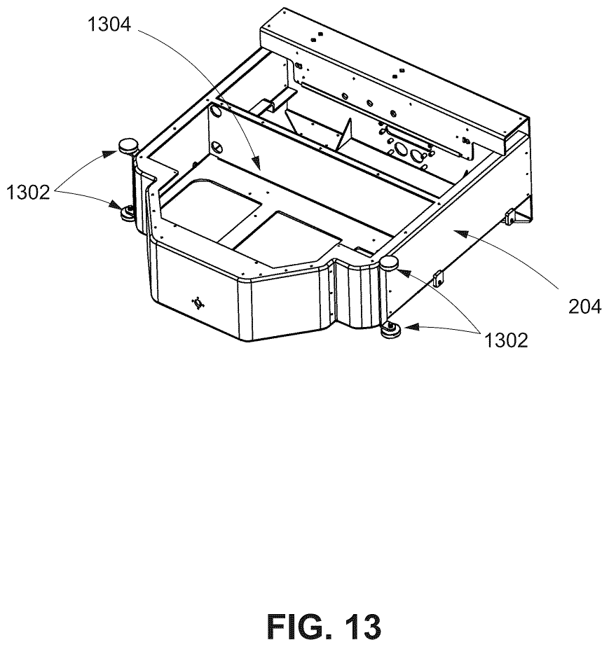

FIG. 13 is a perspective view of the collision mitigation mechanism of an autonomous cleaning robot system in accordance with an embodiment of the invention. In this view, the collision mitigation system includes several rollers 1302, and the rollers 1302 are installed at the edge position of the main mounting frame 204 and protrude from the outer side of the main mounting frame 204. The battery and main controller unit are housed in the middle space 1304 of the frame unit.

In the present embodiment, the main mounting frame 204 is a girder-type complete machine skeleton, and the complete machine has high strength and is relatively simple to manufacture. The above-mentioned spray plate, the roller brush for rolling and mopping the floor, the rolling brush mounting frame, the wringing roller, the fluid receiving tray, the deflector, the filter mechanism, and the suspension frame are jointly made into an integral module. The integral module is assembled on the main mounting frame 204 in a detachable manner. In order to avoid the impact of up and down bumps on the parts when the robot encounters a bumpy field, a shock absorbing structure is provided above each of the drive wheels.

The above-mentioned collision mitigation system includes at least one image capture device for collecting ground image information, and the image capture device sends the collected image information to the main controller for processing to obtain current terrain information. The main controller has pre-stored information of various terrain types (including but not limited to: flat terrain, stepped terrain) and operation instructions corresponding to various types of terrain. The main controller compares the current terrain information with all types of pre-stored terrain features. The terrain information is compared to obtain the current terrain type, and the main control controls the mobile mechanism to execute the operation instruction matching the current terrain type.

In one embodiment of the invention, the sensor technology employs light detection and ranging (Lidar) sensors. The lidar sensors can measure a contour of an object in a two-dimensional scanning plane. The sensors have an effective range of up to 20 meters. Such sensors provide the robot with up to a 270-degree field of detection in front of the robot body. Additional sensors include one or more indoor/outdoor use video cameras. Such cameras employ 1920×1080 pixel resolution at 30 frames per second using. More than one camera enables the robot to stereoscopically map terrain, giving a true three-dimensional means of detecting objects. Persons skilled in the art will readily appreciate that other sensing technologies both known and yet to be known can be employed to provide the autonomous cleaning robot improved functionality.

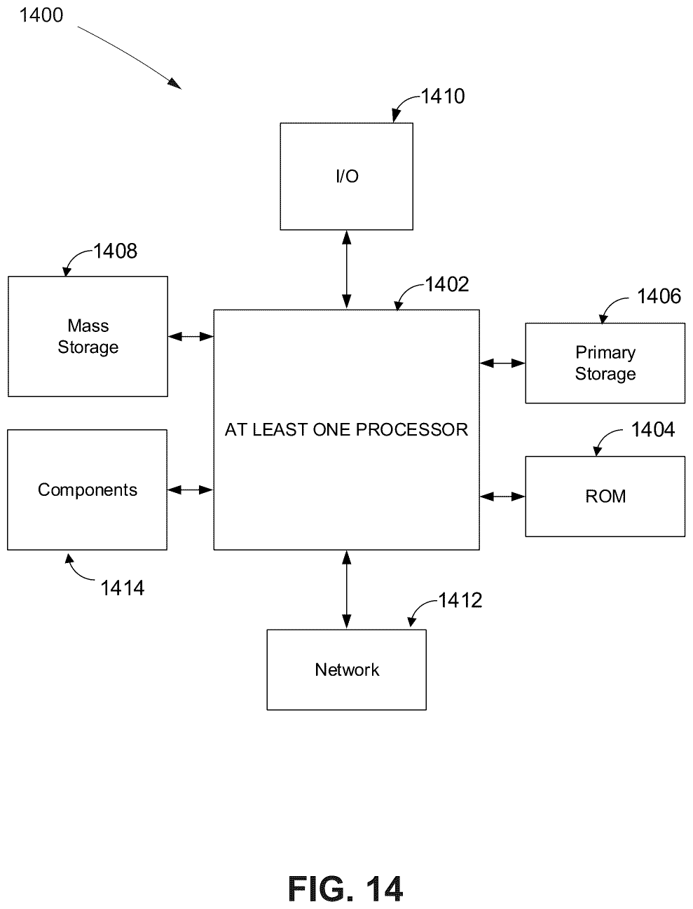

FIG. 14 illustrates a typical computer system that, when appropriately configured or designed, may serve as a computer system for which the main controller unit, and the components thereof, be embodied. The computer system 1400 includes at least one processor 1402 (also referred to as central processing units, or CPUs) that may be coupled to storage devices including a primary storage 1406 (typically a random-access memory, or RAM), a primary storage 1404 (typically a read-only memory, or ROM). The at least one processor or CPU 1402 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general-purpose microprocessors. As is well known in the art, primary storage 1404 acts to transfer data and instructions uni-directionally or bi-directionally to the CPU and primary storage 1406 typically may be used to transfer data and instructions in a bi-directional manner. The primary storage devices discussed previously may include any suitable computer-readable media known and appreciated in the art. A mass storage device 1408 may also be coupled bi-directionally to CPU 502 and provides additional data storage capacity and may include any of the computer-readable media described above. Mass storage device 1402 may be used to store programs, data and the like and typically may be used as a secondary storage medium such as a hard disk or a flash drive. It will be appreciated that the information retained within mass storage device 1408, may, in appropriate cases, be incorporated in standard fashion as part of primary storage 1406 as virtual memory.

The CPU 1402 is coupled to the various components 1410 of the invention such as the drive wheel assembly, the collision mitigation mechanism, the sweeping and vacuum assembly, the cleaning fluid applicator and cleaning roller assembly, and the germicidal ultraviolet light disinfection mechanism. The CPU can also be configured to operate and interpret data the various sensors used by the autonomous cleaning robot. The CPU 1402 may also be coupled to an interface 1410 that connects to one or more input/output devices such as buttons, track balls, mice, keyboards, microphones, touch-sensitive displays, transducer card readers, magnetic or paper tape readers, tablets, styluses, voice or handwriting recognizers, or other well-known input devices such as, of course, other computers. Such interfacing can allow for program modification as well as data gathering. Finally, the CPU 1402 optionally may be coupled to an external device such as a database or a computer, tablet, smartphone, or internet network using an external connection shown generally as a network 1412, which may be implemented as a hardwired or wireless communications link using suitable conventional technologies. With such a connection, the CPU 502 might receive information from a network, or might output information to a network in the course of performing the method steps described in the teachings of the present invention.

It will be understood by persons having skill in the art that memory storing computer readable instructions that, when executed by the at least one processor, cause autonomous cleaning robot system, by at least one processor, to perform the steps of certain functions such as, but not limited to, activating the autonomous cleaning robot; acquiring imagery of the space to be cleaned; checking cleaning fluid levels; activating the sweeping and vacuum assembly; dispensing cleaning fluid through cleaning fluid applicator and roller assembly; recycling recovered and excess cleaning fluid; activating the germicidal ultraviolet light disinfection mechanism to irradiate recovered and excess cleaning fluid, and irradiating a cleaning surface; moving the autonomous cleaning robot across the space to be cleaned; recording the travel path of the autonomous cleaning robot; and housing and powering down the autonomous cleaning robot when the autonomous cleaning robot completes the task of cleaning the surface to be cleaned. It will be further understood by those skilled in the art that other computer readable instructions can be implemented into memory so as to provide a more adaptable and upgradeable cleaning robot.

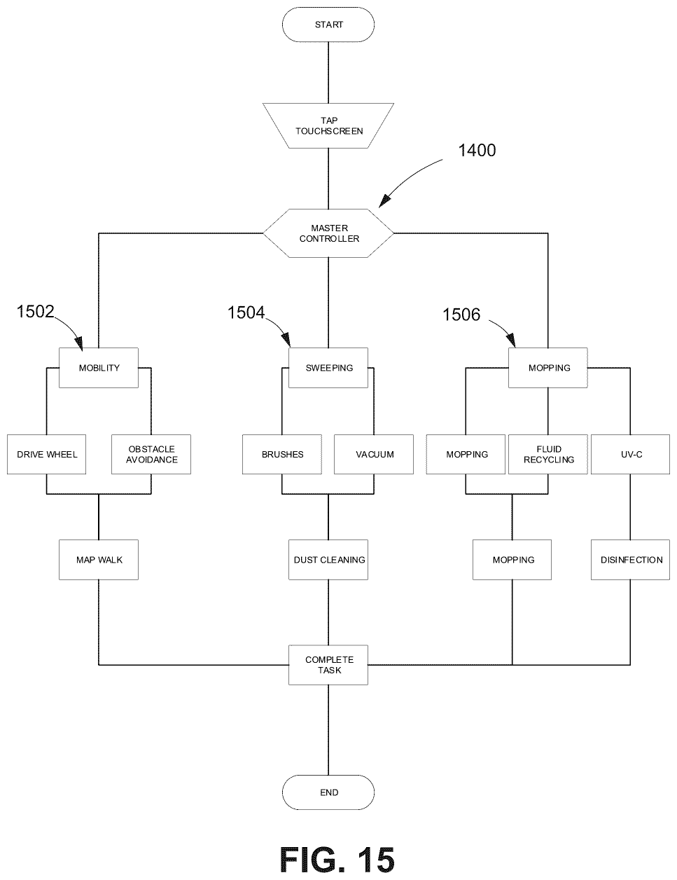

FIG. 15 is a block diagram depicting the general architecture and functionality of an autonomous cleaning robot in accordance with an embodiment of the invention. In this view, the main controller unit or master controller governs the robot's mobility 1502, sweeping 1504, and mopping 1506 mechanisms. In this embodiment, the mobility 1502 functions involve the system's drive wheels and obstacle avoidance systems. The sweeping 1504 functions involve the control of the brushes and vacuum systems. The mopping 1506 functions involve the mopping, fluid recycling, and germicidal UV radiation systems. When in use, these functions operate simultaneously to allow for the autonomous cleaning robot to move about or map walk, to sweep and vacuum dust and debris, and to mop and disinfect a cleaning surface.

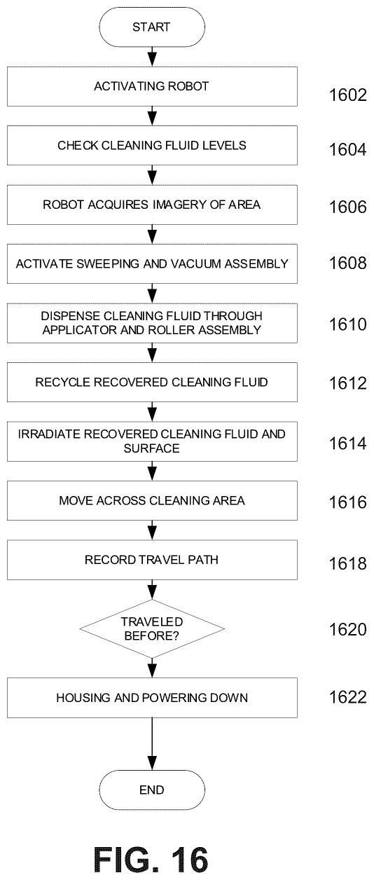

FIG. 16 is a flow diagram of a method for operating an exemplary autonomous cleaning robot system in accordance with an embodiment of the invention. Use of the autonomous is designed to be simple. As a method for cleaning a floor using an autonomous cleaning robot system involves activating the autonomous cleaning robot 1602. The next step involves the checking of cleaning fluid levels 1604. The next step involves the robot system acquiring imagery of the space to be cleaned 1606. The next step involves the robot activating the sweeping and vacuum assembly 1608. The next step involves the robot dispensing cleaning fluid through a cleaning fluid applicator and roller assembly 1610. The next step involves the robot recycling recovered and excess cleaning fluid; the robot activating the germicidal ultraviolet light disinfection mechanism to irradiate recovered and excess cleaning fluid and irradiating a cleaning surface 1612; the autonomous cleaning robot moving across the area or space to be cleaned 1614; the autonomous cleaning robot recording its travel path 1616. The next step involves the autonomous cleaning robot determining whether it has traveled across the same surface and modifying its course to travel across areas not previously traveled across 1618. When the cleaning cycle has completed, the next step involves housing and powering down the autonomous cleaning robot 1620.

Having fully described at least one embodiment of the autonomous cleaning robot system, other equivalent or alternative methods of implementing such an autonomous cleaning robot system and method according to the present invention will be apparent to those skilled in the art. Various aspects of the invention have been described above by way of illustration, and the specific embodiments disclosed are not intended to limit the invention to the particular forms disclosed. The particular implementation of the autonomous cleaning robot system may vary depending upon the particular context or application. By way of example, and not limitation, the autonomous cleaning robot was designed to sweep, vacuum, clean and disinfect flooring. However, similar techniques may instead be applied to other types of cleaning robots, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Although specific features of the autonomous cleaning robot system are shown in some drawings and not others, persons skilled in the art will understand that this is for convenience. Each feature may be combined with any or all of the other features in accordance with the invention. The words “including,” “comprising,” “having,” and “with” as used herein are to be interpreted broadly and comprehensively and are not limited to any physical interconnection. Claim elements and flowchart steps herein may have been numbered and/or lettered solely as an aid in readability and understanding. Any such numbering of flowchart or flow diagram steps is not intended to, and should not be taken to, indicate or limit the ordering of elements and/or steps in the claims to be added at a later date.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. An autonomous cleaning robot comprising:
a. a main body including a frame assembly and an outer shell;
b. a drive wheel assembly;
c. a collision mitigation mechanism;
d. a sweeping and vacuum assembly;
e. a cleaning fluid applicator and cleaning roller assembly;
f. a germicidal ultraviolet light disinfection mechanism, said germicidal ultraviolet light disinfection mechanism capable of disinfecting both cleaning fluid and a cleaning surface; and
g. a main controller unit.

2. The autonomous cleaning robot of claim 1 wherein the sweeping and vacuum assembly comprises a sweeper having two counterrotating brushes, a dust collector, a vacuum cannister, and a suction tube connecting the said dust collector with the said vacuum cannister.

3. The autonomous cleaning robot of claim 1 wherein the cleaning fluid applicator and cleaning roller assembly comprises:
a. a cleaning fluid reservoir;
b. a cleaning fluid outlet pipeline; and
c. a roller brush assembly for cleaning and mopping a floor.

4. The autonomous cleaning robot of claim 3 wherein the cleaning fluid applicator and cleaning roller assembly further comprises a cleaning fluid recycling assembly, said cleaning fluid recycling assembly consisting of:
a. a wringing roller mechanism for wringing a roller brush assembly for cleaning and mopping a floor, said roller mechanism for wringing the roller brush assembly for cleaning and mopping a floor engageable with the said roller brush assembly;
b. a fluid receiving tray;
c. a filtering mechanism for cleaning fluid; and
d. a cleaning fluid pump.

5. The autonomous cleaning robot of claim 3 wherein the cleaning fluid applicator and cleaning roller assembly further comprises:
a. a spray plate assembly;
b. a roller brush; and
c. a roller brush mounting frame.

6. The autonomous cleaning robot of claim 4 wherein the cleaning fluid recycling assembly further comprises a deflector, said deflector positioned between the fluid receiving tray and the roller brush assembly for cleaning and mopping a floor in such a manner that used cleaning fluid can be guided from the roller brush assembly for cleaning and mopping a floor into the fluid receiving tray.

7. The autonomous cleaning robot of claim 1 wherein the germicidal ultraviolet light disinfection mechanism of the cleaning roller assembly includes a UV-C disinfecting lamp, said UV-C disinfecting lamp configured to apply germicidal ultraviolet radiation to the contents inside the fluid reservoir.

8. The autonomous cleaning robot of claim 1 further comprising a suspension mechanism, said suspension mechanism comprising a vertically arranged suspension frame and two sides of the said vertically arranged suspension frame.

9. The autonomous cleaning robot of claim 1 further comprising a terrain navigation system, said terrain navigation system comprising one or more image acquisition devices connected with the main controller unit.

10. An autonomous cleaning robot comprising:
a. a main body including a frame assembly and an outer shell;
b. a drive wheel assembly;
c. a collision mitigation mechanism;
d. a sweeping and vacuum assembly;
e. a cleaning fluid applicator and roller assembly;
f. a germicidal ultraviolet light disinfection mechanism capable of disinfecting both cleaning fluid and a cleaning surface;
g. a cleaning fluid recycling assembly; and
h. a main controller unit.

11. The autonomous cleaning robot of claim 10 wherein the sweeping and vacuum assembly comprises a sweeper including two counterrotating brushes, a dust collector, a vacuum cannister, and a suction tube connecting the said dust collector with the said vacuum cannister.

12. The autonomous cleaning robot of claim 10 wherein the cleaning fluid applicator and roller assembly comprises:
a. a cleaning fluid reservoir;
b. a cleaning fluid outlet pipeline; and
c. a roller brush assembly for cleaning and mopping a floor.

13. The autonomous cleaning robot of claim 10 wherein the cleaning fluid recycling assembly comprises:
a. a wringing roller mechanism for wringing a roller brush assembly for cleaning and mopping a floor, said roller mechanism for wringing the roller brush assembly for cleaning and mopping a floor engageable with the said roller brush assembly;
b. a fluid receiving tray;
c. a filtering mechanism for cleaning fluid; and
d. a cleaning fluid pump.

14. The autonomous cleaning robot of claim 10 wherein the cleaning fluid recycling assembly further comprises a deflector, said deflector positioned between the fluid receiving tray and the roller brush assembly for cleaning and mopping a floor in such a manner that used cleaning fluid can be guided from the roller brush assembly for cleaning and mopping a floor into the fluid receiving tray.

15. The autonomous cleaning robot of claim 12 wherein the cleaning fluid applicator and roller assembly further comprises:
a. a spray plate assembly;
b. a roller brush; and
c. a roller brush mounting frame.

16. The autonomous cleaning robot of claim 1 wherein the germicidal ultraviolet light disinfection mechanism of the cleaning roller assembly includes a UV-C disinfecting lamp, said UV-C disinfecting lamp configured to apply germicidal ultraviolet radiation to the contents inside the fluid reservoir.

17. The autonomous cleaning robot of claim 10 further comprising a suspension mechanism, said suspension mechanism comprising a vertically arranged suspension frame and two sides of the said vertically arranged suspension frame.

18. The autonomous cleaning robot of claim 10 further comprising a terrain navigation system, said terrain navigation system comprising one or more image acquisition devices connected with the main controller unit.

19. The autonomous cleaning robot of claim 10 wherein the main controller unit comprises at least one processor and memory, said memory including computer executable instructions which, when executed by the said at least one processor, cause the autonomous cleaning robot system, by at least one processor, to perform the steps of:
a. activating the autonomous cleaning robot; acquiring imagery of the space to be cleaned;
b. checking cleaning fluid levels; activating the sweeping and vacuum assembly; dispensing cleaning fluid through cleaning fluid applicator and roller assembly;
c. recycling recovered and excess cleaning fluid in the cleaning fluid recycling assembly;
d. activating the germicidal ultraviolet light disinfection mechanism to irradiate recovered and excess cleaning fluid, and irradiating a cleaning surface;
e. moving the autonomous cleaning robot across the space to be cleaned;
f. recording the travel path of the autonomous cleaning robot; and
g. housing and powering down the autonomous cleaning robot when the autonomous cleaning robot completes the task of cleaning the surface to be cleaned.

20. A method for operating an autonomous cleaning robot comprising a main body including a frame assembly and an outer shell; a drive wheel assembly; a collision mitigation mechanism; a sweeping and vacuum assembly; a cleaning fluid applicator and roller assembly; a germicidal ultraviolet light disinfection mechanism capable of disinfecting both cleaning fluid and a cleaning surface; a cleaning fluid recycling assembly; and a main controller unit; the method comprising the steps of:
a. activating the autonomous cleaning robot;
b. acquiring imagery of the space to be cleaned;
c. checking cleaning fluid levels;
d. activating the sweeping and vacuum assembly;
e. dispensing cleaning fluid through cleaning fluid applicator and roller assembly;
f. recycling recovered and excess cleaning fluid;
g. activating the germicidal ultraviolet light disinfection mechanism to irradiate recovered and excess cleaning fluid, and irradiating a cleaning surface;
h. moving the autonomous cleaning robot across the space to be cleaned;
i. recording the travel path of the autonomous cleaning robot; and
j. housing and powering down the autonomous cleaning robot when the autonomous cleaning robot completes the task of cleaning the surface to be cleaned.
